(19) United States
(12) Patent Application Publication
(10) Pub. No.: US 2006/0207597 A1
(43) Pub. Date: Sep. 21, 2006

(54) Adjustable bite block for respirator

(76) Inventor: Clifford A. Wright, San Diego, CA (US)

Correspondence Address: PERKINS COIE LLP, POST OFFICE BOX 1208, SEATTLE, WA, 98111-1208, US

(21) Appl. No.: 11/375,682

(22) Filed: Mar. 14, 2006

Related U.S. Application Data
(60) Provisional application No. 60/662,177, filed on Mar. 15, 2005.

(51) Int. Cl. A62B 7/10; A61M 16/00; A61M 15/08

(52) U.S. Cl. 128/206.11; 128/200.26; 128/206.12; 128/207.18; 128/859; 128/861

(57) ABSTRACT

A respiration device includes an adjustable nasal feed path and a bite block that is configured to be coupled to the mouth of a patient. The respiration device includes a mouth piece optionally having an internal one-way valve. The mouth piece is attached to a pair of nasal conduits that are adjustable for comfortably fitting in the patient's nostrils.

### PRIORITY CLAIM

This application claims priority to U.S. Provisional Patent Application No. 60/662,177, filed Mar. 15, 2005, which is incorporated herein by reference.

### BACKGROUND

A variety of respiratory devices, such as masks and bite blocks, are known. The devices typically include one or more components that couple with a patient's mouth and one or more devices that are coupled to the patient's nostrils. The devices are used to provide variable airway pressure, which varies with the patient's respiratory cycle or which varies with the condition of the patient, such as to treat a medical disorder. For example, positive airway pressure can be provided to the mouth while negative airway pressure is provided to the nostrils.

The portion of the device that couples to the nostrils can comprise nasal pillows. The nasal pillows are fixedly attached to the bite block and are positioned so as to be readily insertable into the patient's nostrils when the bite block is positioned in the patient's mouth. However, due to the variations in the sizes and shapes of patients' faces, it can be difficult to provide a respiratory device that fits comfortably on a specific patient. As a result, the patient typically has to deal with some level of discomfort. This is undesirable, as the patient typically wears the device for an extended period of time. Consequently, the patient may strongly avoid wearing an uncomfortable mask, which defeats the purpose of the therapy.

In view of the foregoing, there is a need for a respiratory device that can be adjusted to the particular size and shape of a patient's face.

### SUMMARY

A respiration device includes an adjustable nasal feed path and a bite block that is configured to be coupled to the mouth of a patient. The respiration device includes a mouth piece optionally having an internal one-way valve. The mouth piece is attached to a pair of nasal conduits that are adjustable for comfortably fitting in the patient's nostrils.

Other features and advantages of the invention will appear hereinafter. The features of the invention described above can be used separately or together, or in various combinations of one or more of them. The invention resides as well in sub-combinations of the features described. Furthermore, many of the method steps described herein may be performed in a different order than that which is explicitly described.

### BRIEF DESCRIPTION OF THE DRAWINGS

FIG. 1 is a rear, perspective view of a respiratory device.

FIG. 2 is a front, perspective view of the respiratory device.

FIG. 3 is a rear, exploded view of the respiratory device.

FIG. 4 is a front view of the device in phantom.

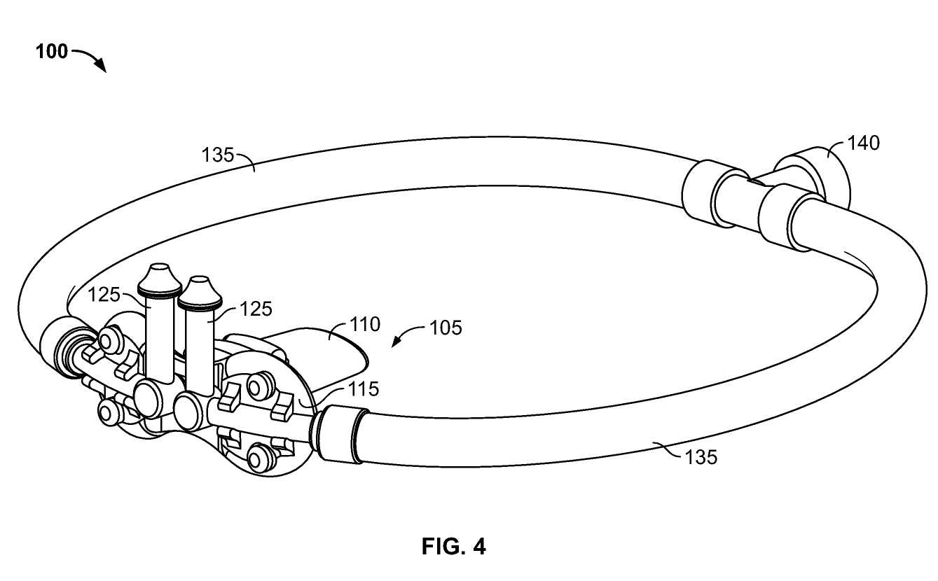

Like reference symbols in the various drawings indicate like elements.

### DETAILED DESCRIPTION OF THE DRAWINGS

Various embodiments of the invention will now be described. The following description provides specific details for a thorough understanding and enabling description of these embodiments. One skilled in the art will understand, however, that the invention may be practiced without many of these details. Additionally, some well-known structures or functions may not be shown or described in detail so as to avoid unnecessarily obscuring the relevant description of the various embodiments.

The terminology used in the description presented below is intended to be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this detailed description section.

Where the context permits, singular or plural terms may also include the plural or singular term, respectively. Moreover, unless the word “or” is expressly limited to mean only a single item exclusive from the other items in a list of two or more items, then the use of “or” in such a list is to be interpreted as including (a) any single item in the list, (b) all of the items in the list, or (c) any combination of items in the list.

Disclosed is a respiration device with an adjustable nasal feed path. The respiration device has a mouth piece with an internal one-way valve. The mouth piece is attached to a pair of nasal conduits that are adjustable to the patient, as described in detail below.

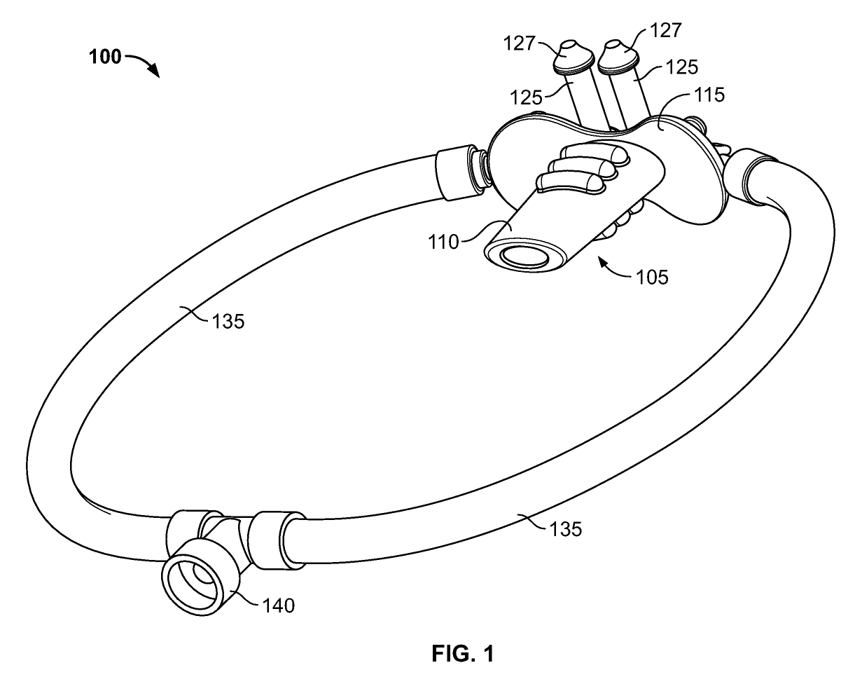

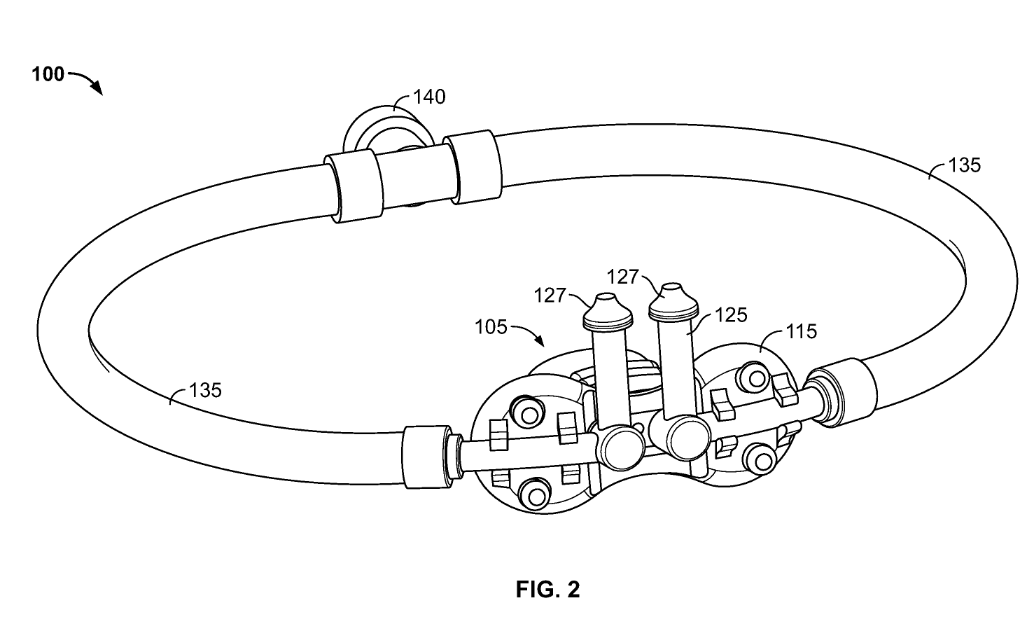

With reference to FIGS. 1 and 2, the device 100 includes a bite block 105 that is configured to be coupled to the mouth of a patient. For example, the bite block 105 can be shaped to be positioned at least partially within the mouth of a patient or can be shaped to be positioned over the mouth of the patient. In the illustrated embodiment, the bite block 105 has a mouth region 110 that fits inside the mouth of a patient. The mouth region 110 can have any of a variety of shapes configured to fit comfortably in a patient's mouth. In the illustrated embodiment, the mouth region 110 is oblong and tapers in cross-sectional size moving inwardly relative to the mouth. A fluid pathway extends through the mouth region 110 for providing fluid flow to the patient's mouth when the bite block 105 is positioned in the mouth. The fluid pathway defines an opening through the distal end of the mouth region 110.

The bite block 105 further includes an outer flange 115 that is coupled to the mouth region 110. The outer flange 115 rests against the patient's face (such as against the patient's lips) when the patient us using the bite block 105. As described in detail below, a one-way valve is disposed inside the bite block 105 along the fluid pathway for the regulation of fluid flow to or from the patient's mouth. For example, the one-way valve may be positioned such that it permits fluid flow only in an expiratory direction. In this manner, the bite block 105 can be used to limit the patient to only exhaling through his or her mouth.

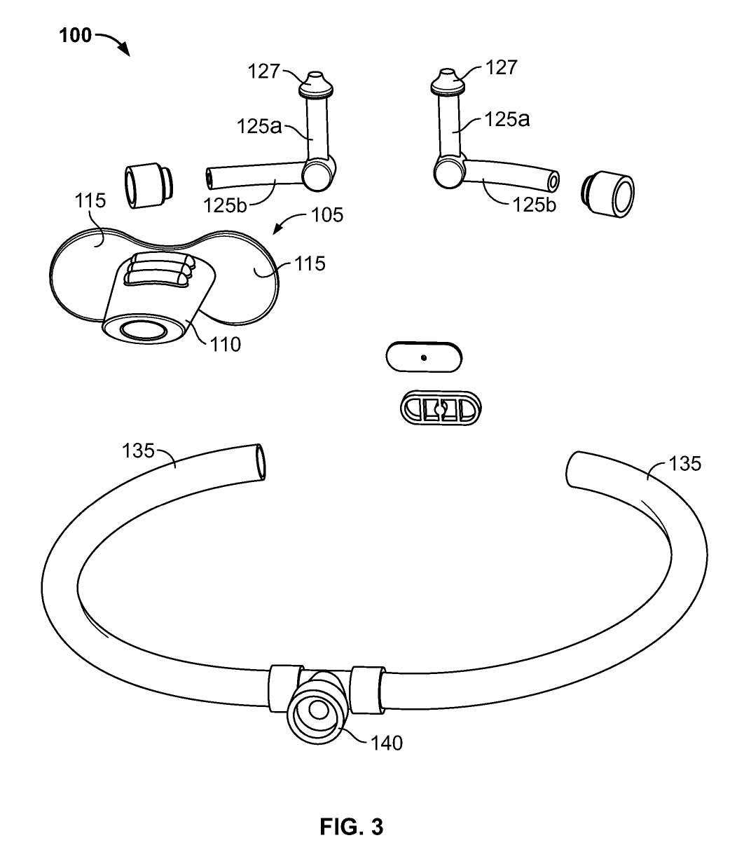

As best shown in FIG. 2, a pair of nasal conduits 125 is coupled to the bite block 105. The nasal conduits 125 comprise a pair of tubes that extend upwardly or outwardly from the bite block 105 and that are configured to be positioned inside of the patient's nostrils. In this regard, each nasal conduit 125 includes an upward-extending region 125a that points upward toward the patient's nostril for insertion into the patient's nostrils. The upward-extending region 125a may include a pillow 127 for providing a secure fit with the internal region of the nostril, as best shown in the exploded view of FIG. 3. The pillow 127 can be shaped to provide a comfortable fit with the internal region of the patient's nostril and to seal with the nostril. The shape of the pillow 127 may vary.

The nasal conduits 125 further include a transverse region 125b (FIG. 3) that extends transversely to the upward-extending region 125a and that is adjustably mounted to the bite block 105 using one or more swivel adapters or other suitable connectors. In one embodiment, the nasal conduits 125 are adjustable upward and outward to be movable into an optimal position relative to the size of the patient's face and the position of the patient's nostrils. The configuration of the nasal conduit 125 may vary. For example, the nasal conduit 125 may comprise a main tube that forks into separate branch tubes for insertion into each nostril. The nasal conduits 125 may also have various shapes that are configured to extend toward the patient's nostrils when the bite block 105 is positioned in the patient's mouth. Regardless of the shape, the nasal conduit 125 is adjustable to the patient. The nasal conduit 125 may be linearly adjustable along one or more axes and can also be rotatably adjustable about one or more axes.

With reference still to FIGS. 1 and 2, one or more fluid pathways 135 are coupled to or in fluid communication with the bite block 105 and the nasal conduit 125. The fluid pathways 135 comprise one or more tubes that are coupled to an interface 140 that can be attached to a source of fluid flow, such as a device for assisting respiration of the patient. In the illustrated embodiment, the fluid pathways 135 comprise a pair of curved tubes that are attached to the interface 140. It should be appreciated that the fluid pathways can take on a variety of shapes and sizes.

In use, the device 100 is coupled to the patient by inserting the mouth region 110 of the bite block 105 into the patient's mouth. As discussed, the mouth region 110 has a shape and size that facilitates insertion into the patient's mouth in a relatively comfortable manner. When the mouth region 110 is in the patient's mouth, the outer flange 115 rests against the outer surface of the patient's lips to provide a level of sealing. As mentioned, the bite block 105 may have other shapes that are configured for coupling to the patient's mouth.

The nasal conduits 125 are next inserted into the patient's nostrils such that each nostril couples with a corresponding conduit 125. As discussed, the conduits 125 may optionally include enlarged pillow regions 127 for providing a secure fit within the nostrils and sealing with the nostrils. Advantageously, the orientation of the nasal conduits 125 are adjustable relative to the patient's nostrils and the bite block, such as in upward and outward directions. In this manner, the nasal conduits 125 can be adjusted to facilitate comfort of the patient and to also facilitate a secure coupling with the nostrils. The nasal conduits can also be rotatable about one or more axes to permit further adjustment of the orientation and positioning of the nasal conduits relative to the nostrils. Once the nasal conduits are coupled with the patient's nostrils, the patient or an assistant can then provide further adjustment to the orientation and position of the nasal conduits to further refine the nasal conduit positioning and provide increased comfort to the patient.

The interface 140 is then attached to a fluid source or a source of respiratory assistance for providing fluid flow to the air pathways 135 that are coupled to the bite block 105 and the nasal conduits 125. This can be used to provide positive or negative airway pressure to the nose and to the mouth, such as to assist in respiration.

Fluid can flow in an inspiratory or an expiratory direction through the fluid pathways 135. As mentioned, the bite block 105 includes a one-way valve that is positioned in the fluid pathway that extends through the mouth region 110 of the bite block 105. The one-way valve is positioned so as to limit fluid to flowing only in a desired direction through the mouth. For example, the one-way valve can be positioned to limit fluid to flowing only in an expiratory direction through the bite block. Thus, the patient is limited to exhaling through the mouth when the bite block 105 is positioned in the patient's mouth. The patient can inhale through the nasal conduits 125.

While specific embodiments have been shown and described, various changes and substitutions may of course be made, without departing from the spirit and scope of the invention. The invention, therefore, should not be limited, except by any claims and their equivalents.

What is claimed is:

1. A respiration device, comprising:
a bite block configured for coupling with a mouth of a patient; and
a plurality of nasal conduits coupled to the bite block for insertion into nostrils of the patient, with the nasal conduits adjustable in a plurality of directions.

2. The respiration device of claim 1 wherein a fluid pathway is defined in the bite block for providing fluid flow to or from the mouth of the patient.

3. The respiration device of claim 1 wherein the bite block includes an oblong, tapered region for insertion in the mouth of the patient.

4. The respiration device of claim 1 wherein the bite block includes an outer flange for resting against a face of the patient.

5. The respiration device of claim 1 further comprising a one-way valve disposed inside the bite block for regulating fluid flow through the mouth of the patient.

6. The respiration device of claim 1 further comprising a pillow on a distal end of each nasal conduit for providing a secure fit within the nostril of the patient.

7. The respiration device of claim 1 wherein each of the nasal conduits includes an upward-extending region for insertion into the nostril of the patient, and a transversely-extending region adjustably mounted to the bite block.

8. The respiration device of claim 7 wherein the transversely-extending region is adjustably mounted to the bite block via a swivel adapter.

9. The respiration device of claim 1 wherein each of the nasal conduits is linearly adjustable along at least one axis.

10. The respiration device of claim 1 wherein each of the nasal conduits is rotatably adjustable about at least one axis.

11. The respiration device of claim 1 wherein each of the nasal conduits is adjustable in an upward direction and an outward direction relative to the bite block.

12. The respiration device of claim 1 wherein the plurality of nasal conduits comprises a main tube that forks into a plurality of branch tubes for insertion into the nostrils of a patient.

13. The respiration device of claim 1 further comprising a tube, which defines a fluid pathway, coupled to at least one of the bite block and the nasal conduits.

14. The respiration device of claim 13 further comprising an interface coupled to the tube, with the interface configured for attachment to a fluid flow source.

15. A respiration device, comprising:
a bite block including a region for insertion into a mouth of a patient; and
a plurality of nasal conduits coupled to the bite block for insertion into nostrils of the patient, wherein each of the nasal conduits is linearly adjustable along at least one axis, and rotatably adjustable about at least one axis.

16. The respiration device of claim 15 wherein each of the nasal conduits is adjustable in an upward direction and an outward direction relative to the bite block.

17. The respiration device of claim 15 further comprising a one-way valve disposed inside the bite block for regulating fluid flow through the mouth of the patient.

18. The respiration device of claim 15 wherein each of the nasal conduits includes an upward-extending region for insertion into the nostril of the patient, and a transversely-extending region adjustably mounted to the bite block.

19. The respiration device of claim 15 further comprising:
a tube, which defines a fluid pathway, coupled to at least one of the bite block and the nasal conduits; and
an interface coupled to the tube, with the interface configured for attachment to a fluid flow source.

20. A respiration device, comprising:
first means for providing a fluid pathway to or from a mouth of a patient; and
second means, coupled to the first means, for providing a fluid pathway to or from nostrils of the patient, wherein the second means is linearly adjustable along at least one axis, and rotatably adjustable about at least one axis.
